(19) United States
(12) Patent Application Publication
(10) Pub. No.: US 2006/0134718 A1
(43) Pub. Date: Jun. 22, 2006

(54) Assay for camp based on exclusive binding of camp to the b-binding sites of camp dependent protein kinase (capk)

(76) Inventor: Stein Ove Doskeland, Bergen (NO)

Correspondence Address: SUGHRUE MION, PLLC, 2100 PENNSYLVANIA AVENUE, N.W., SUITE 800, WASHINGTON, DC, 20037, US

(21) Appl. No.: 10/528,560

(22) PCT Filed: Sep. 22, 2003
(86) PCT No.: PCT/GB03/04044

§ 371 Date: Oct. 4, 2005

(30) Foreign Application Priority Data
Sep. 20, 2002 (GB) 0221915.2

(51) Int. Cl. C12Q 1/42

(52) U.S. Cl. 435/21

(57) ABSTRACT

The present invention provides a method for assaying for cAMP in a sample, said method comprising contacting a sample with an unknown cAMP content with a polypeptidic cAMP binding agent and optionally with a labelled cAMP and detecting conjugates of cAMP or labelled cAMP and said binding agent, characterized in that said binding agent comprises functional cAPK cAMP B-binding sites only.

This invention relates to a method for assaying for cyclic adenosine monophosphate (cAMP) and other cyclic nucleotides and cyclic nucleotide analogs and to kits for use in such methods.

cAMP or adenosine 3′,5′-cyclic monophosphate comprises a ribofuranose monosaccharide unit with a purine group attached at the 1′ position and a phosphate group attached at the 3′ and 5′ positions. cAMP is formed by cyclization of ATP and is a “second messenger” which functions as a mediator of hormone action for a range of hormones. In particular it serves to activate key enzymes known as protein kinases.

cAMP is widely distributed in the body and has been assayed for in connection with a broad range of conditions, eg thyroid malfunction, calcium metabolism disorders, etc.

Several assays for cAMP are available commercially, especially from Amersham plc, UK. The most widely used assays are based on competition between cAMP and a labelled (eg radiolabelled) cAMP or cAMP analog for a high affinity binding site either on an immunoglobulin or on a naturally occurring cAMP binding protein, in particular cAMP dependent protein kinase isoenzyme type I (cAPKI).

While such assays have been available for some decades now, they are nonetheless less accurate than is desirable and the antibody based assays are unduly sensitive to the agents (eg EDTA) that are added to cAMP containing samples to prevent degradation of cAMP before the assay can be carried out.

There is thus a need for an improved assay for cAMP.

We have now found that such an improved assay can be achieved by using as a cAMP binding agent a polypeptide comprising in functional form only one type of cAMP binding site of a cAMP dependent protein kinase (cAPK). cAPK, which exists as type I (cAPKI) and type II (cAPKII), has two types of cAMP binding sites, the A and B sites, on each of the regulatory (R) subunits located respectively towards and at the C-terminus. Thus for cAPKI, which is a dimer, there are four binding sites Aα, Aβ, Bα and Bβ. In terms of amino acid sequence the A sites on the α and β chains are identical as are the B sites on the α and β chains.

cAMP dependent protein kinases are discussed for example in Taylor et al. Ann. Rev. Biochem. 59: 971 (1990), Doskeland et al. Biochim. Biophys. Acta 1178: 249-258 (1993), Francis et al. Ann. Rev. Physiol. 56: 237-272 (1994) and Johnson et al. Chem. Rev. 101: 2243-2270 (2001). The A and B sites of the polypeptide RIα (the regulatory (R) subunit of the a chain of cAMP dependent protein kinase type I) is described by Yu et al. in Science 269: 807-813 (1995) who co-crystallized cAMP and a truncated RIα with intact A and B sites. RI has two forms, RIα and RIβ which are very similar in terms of amino acid sequence in the cAMP binding domains. The R subunits of cAMP dependent protein kinase type II (i.e. RIIα and RIIβ) have A and B sites that are similar to the corresponding sites in RIα. (See Diller et al. Structure 9: 73-82 (2001)).

In mammalian R subunits, the A site comprises roughly amino acid residues 143-260 (RIα) or 158-277 (RIIβ) and the B site comprises roughly residue 260-374 (RIα) or 277-426 (RIIβ).

Thus viewed from one aspect the invention provides a method for assaying for cAMP in a sample, said method comprising contacting a sample with an unknown cAMP content with a polypeptidic cAMP binding agent and optionally with a labelled cAMP, and detecting conjugates of cAMP or labelled cAMP and said binding agent, characterized in that said binding agent comprises functional cAPK cAMP B-binding sites only.

The functional site is a B site, more preferably a RI B-site, most preferably a RIα B-site.

By “functional site” is meant herein that the binding site is capable of binding cAMP with a K_{D} of no more than 300% of that of the site in native human cAPK, preferably no more than 200%, more preferably no more than 150% and especially no more than 110% . (The lower the K_{D}, the higher the binding affinity).

Besides the functional cAMP binding site (or sites), the binding agent may contain non-functional cAMP binding sites, e.g. of a different type. By a non-functional site is meant a site corresponding to a native cAMP binding site in some or all of its amino acid sequence but which has a K_{D} for cAMP binding which is more than 300% of that of the corresponding site in native human cAPK, preferably more than 500% and more preferably more than 10000%.

By a “cAPK cAMP binding site” is meant a polypeptide sequence having the ability to bind cAMP and having a sequence homology with a native mammalian cAPK cAMP binding site sequence of at least 60%, preferably at least 80%, most preferably at least 95%. The cAPK cAMP binding site in the binding agent used in the assay method of the invention thus need not be identical to a native mammalian cAPK cAMP binding site, e.g. due to insertions, deletions, substitutions or transpositions of one or more amino acids. The cAPK cAMP binding site in the binding agent will preferably contain at least 95% of the amino acid sequence of the corresponding cAPK cAMP binding site common to human and bovine cAPK, i.e. the highly conserved section of the sequence. The term “functional cAPK cAMP binding site” is also considered to extend to cAMP binding polypeptide sequences having a functional topology equivalent to native cAPK cAMP binding sites, eg synthetic polypeptide sequences having similar tertiary structure presenting the same or equivalent functional groups within the cAMP binding pocket.

As is routine in diagnostic assays, the detection of the analyte:binding agent conjugate may be direct or indirect. Thus for example where labelled cAMP is used in the assay method a signal from the label in the labelled cAMP:binding agent conjugate, for example a radiation emission, may be detected directly and the content of cAMP in the sample inferred from this. Alternatively the signal from the label in the uncomplexed labelled cAMP may be detected directly and the content of cAMP in the sample again inferred from this. As a further alternative, a secondary binding agent which binds to cAMP:primary binding agent conjugates or to uncomplexed primary binding agent and which gives rise to a directly detectable signal or event may be used and from the detected signal the content of cAMP in the sample may again be inferred. All such systems of analyte detection are encompassed by the assay method of the invention.

Viewed from a further aspect the invention also provides a kit for a cAMP assay, said kit comprising a polypeptidic primary binding agent capable of binding cAMP; optionally, a labelled cAMP; and optionally a secondary binding agent; characterized in that said primary binding agent comprises functional cAPK cAMP B-binding sites only. The kit of the invention also preferably includes instructions for the performance of the assay method.

Viewed from a still further aspect the invention provides a polypeptidic cAMP binding agent which comprises functional cAPK cAMP B-binding sites only, and compositions and items comprising said binding agent.

By compositions containing the binding agent are included liquid, semi-solid (e.g. gel) and solid (e.g. pulverulent) compositions, typically including further substances such as liquid or solid carriers or gel forming agents, preservatives, pH modifiers, etc. By items comprising the binding agent are included for example solid or semi-solid structures coated or impregnated with the binding agent, e.g. beads, plates, tubes, membranes, fibres, etc with the binding agent immobilized thereon.

The cAMP binding agent used according to the invention is preferably a recombinant protein comprising one or more, e.g. 1, 2 or 3, cAPK cAMP binding sites, especially preferably bovine cAPK cAMP binding sites, optionally a fusion protein comprising a section couplable to a substrate surface (a “surface binding region”). Where the binding agent contains two or more cAPK cAMP binding sites, these should be of the same type, thus for example both or all may be RIα B-sites. The protein may, but need not necessarily, include a disabled cAPK cAMP binding site. By disabled in this context is meant that the amino acid sequence of the cAMP binding site is modified to substantially reduce or eliminate the cAMP binding ability of the site, e.g. by substitution, insertion or deletion of one or more amino acids so as to change the tertiary structure or the functional topology of the site. In a preferred embodiment, the binding agent thus has one functional RI B-site and one disabled RI A-site. A-site disablement is conveniently achieved by mutagenesis replacing a highly conserved Gly residue (depending on the species this may be residue 199, 200 or 201) in mammalian, e.g. human or bovine, RIα by Glu. This can be done using PCR and a standard mutagenesis kit (e.g. Strategene Quick Change). The efficacy of A-site disablement can be verified by equilibrium binding, e.g. using the method of Hummel et al. BBA 63: 530-532 (1962) or by binding of tritiated cAMP and precipitation with ammonium sulphate as described further below. Especially preferably the binding agent is a chimeric fusion protein containing one or more cAPK B-sites, especially RIα B-sites, and a polypeptide region which is one member of a specific binding partner pair (e.g. a biotin binding site of streptavidin) or which is a surface binding extension (e.g. Gly Gly Cys or GST (glutathione S-transferase)). Such chimeric fusion proteins comprising disabled cAPK A-sites and one or more cAPK B-sites may readily be prepared by conventional techniques, eg transfection of bacterial hosts such as E. coli with plasmids incorporating in an operable reading frame an appropriately coded nucleic acid sequence. In an especially preferred embodiment of the invention the binding agent comprises a B-site modified relative to a native mammalian cAPK B-site by replacement of Cys by Ala or by another amino acid residue that cannot form a disulphide bridge. This can be done by mutagenesis as discussed above in relation to A-site disablement. Such binding agents containing a Cys-free B-site are more stable on storage than binding agents with Cys residues in the B-site.

The binding agent is preferably used in aqueous solution under non-oxidizing conditions; typically the solution will contain one or more chelating agents such as EDTA and EGTA as stabilizers, and a reducing agent such as DTE, DTT, 2-mercaptoethanol or glutathione.

Where the assay method of the invention involves competition for binding between the cAMP in the sample and a labelled cAMP, it is preferred that the label be tritium (³H) and/or that the label be attached to a substituent at the 8-position, i.e. as a substituent on the adenosine ring structure. cAMP 8-labelled with ¹²⁵I (generally attached via a linker group) is novel and forms a further aspect of the present invention.

³H-cAMP is available commercially from Amersham plc, UK.

The label in the labelled cAMP may be any species which is detectable (e.g. by virtue of radiation emission or absorption) or which can generate a detectable species or event (e.g. by virtue of enzymatic activity). Radiolabels, chromophores and fluorophores are preferred as the label may be placed so as not to interfere with or to minimally interfere with the ability of the labelled cAMP to bind to the cAMP binding agent. In one embodiment of the invention the labelled cAMP is surface-bound cAMP with the conjugates formed by binding of the cAMP binding agent to the surface-bound cAMP being detectable for example by surface plasmon resonance. Such surface binding of cAMP may be effected for example by reacting a substrate having surface carboxyls or activated carboxyls with an 8-aminoalkanoylamino-cAMP (e.g. 8-aminooctanoyl amino-cAMP, available from BioLog, Bremen, Del.). In this embodiment the linker between the surface (e.g. the polymer backbone of a polymeric substrate) and the pendant cAMP groups preferably has a molecular weight of less than 1000D, more preferably a molecular weight of 100 to 500D. (In this way the SPR signal difference on binding of the binding agent to the surface is optimised.) In another embodiment of the invention the labelled cAMP is a polymer (e.g. a dendrimer) with pendant cAMP groups (preferably 8-attached). Labelled cAMP:binding agent conjugates can in this embodiment be detected by light scattering (e.g. turbidimetry or nephelometry). Where the labelled cAMP is radiolabelled it is preferably ³H-cAMP (detectable by scintillation) or 8-(¹²⁵I-X)-cAMP (where X is an organic group providing a chain of up to 25 atoms to link the iodine to the 8-position, e.g. a tyrosine-alkanoylamino group). Alternatively ³²P-labelled cAMP (available from ICN Pharmaceuticals) may be used. Preparation of labelled cAMP can be effected by conventional chemical synthesis techniques. Thus for example 8-amino-octanoyl-amino cAMP (available from BioLog, Bremen, Del.) may be reacted with tyrosine to introduce a tyrosine residue at the 8-position. This can be iodinated with ¹²⁵I using a standard kit (e.g. operable to effect peroxidase catalysed iodination using an inorganic ¹²⁵I compound). The bulky substitution of the 8-position does not appear to inhibit binding of the cAMP to a cAPK cAMP binding site.

In one especially preferred embodiment of the invention, conjugates of cAMP and of labelled cAMP with the cAMP binding agent are precipitated and taken up by a filter, e.g. a membrane filter. In this embodiment, the conjugate precipitation is conveniently effected using a sulphate solution, e.g. aqueous ammonium sulphate. The cAMP binding sites of cAPK do allow for cAMP exchange (i.e. complex dissociation) and so it is desirable to “freeze” the quantity of labelled cAMP in the precipitate. This can readily be done by the use of a precipitation agent comprising a solution of cAMP and sulphate. The solution is preferably almost sulphate saturated, e.g. 55-95% saturated, more especially 75-92% saturated and preferably contains cAMP in a quantity in excess of the labelled cAMP in the sample to which it is to be added, eg at a 0.1 mM concentration. In general this “stop” solution will be added cold in a volume ratio of about 20:1 to 1:2, more preferably about 15:1 to 5:1 relative to the sample. Typically the stop solution is applied at temperatures below 10° C., e.g. 0° C., and if there is a delay between stop solution application and separation of the precipitated conjugate from the supernatant, the sample should be held cold during this delay period, e.g. at 0° C. The stop solution is preferably buffered, e.g. at pH7.

In this embodiment, following conjugate precipitation, the precipitate and supernatant are preferably separated by filtration, e.g. over a micrometer range pore size filter (e.g. 0.45 μm pore size). Suitable filter membranes are available commercially from Millpore, e.g. HAWP or more preferably HAMK filters.

After filtration, the filter membrane is preferably rinsed to remove uncomplexed labelled cAMP. This is preferably done immediately after filtration and is preferably repeated, e.g. one or two further rinses. Diluted stop solution may be used as the rinse solution; generally however this need not contain cAMP.

Where the sample has a high salt content, filtration should be effected as soon as possible after precipitation, e.g. within 30 minutes.

Where the sample under investigation has a low binding agent concentration, it is desirable to add casein to the stop solution to promote precipitation. Where the sample under investigation has a high binding agent concentration, it may be desirable to use glass fibre pre-filters.

Following precipitation and rinsing, the quantity of labelled cAMP retained by the filter membrane may be detected. In the case of chromophore or fluorophore labelling or labelling with radiolabels other than those requiring scintillation counting, the signal may be read from the membrane or following release of the conjugate or the labelled cAMP from the membrane. Where detection involves scintillation counting, e.g. as with tritium labelled cAMP, the conjugate or the labelled cAMP must be released from the membrane. This may conveniently be effected by contacting the membrane with an aqueous surfactant solution, e.g. a sodium dodecyl sulphate solution, typically a 1-5% w/v solution, especially a 2% w/v solution. The conjugate or the labelled cAMP released into solution may then be detected in the solution, e.g. in the case of ³H-cAMP by addition of a scintillation fluid (e.g. “Emulsifier safe for aqueous samples” available from Packard).

As with most diagnostic assays, the assay system for use according to the invention will generally require calibration against standards (i.e. cAMP solutions) with a range of known cAMP concentrations. The assay kit of the invention will thus typically be supplied with a set of standards for calibration and/or a calibration chart and/or a computer program or dataset for interpolation or extrapolation from signal values for standards to determine the cAMP content of the sample under investigation.

The sample investigated using the method of the invention will typically be of a biological or biologically derived material, e.g. microorganisms, cells, tissues, body fluids, body organs, etc. Of particular interest are samples of or derived from microorganisms, especially bacteria and yeasts. Also of interest are samples of or derived from multicellular organisms, e.g. mammals, reptiles, birds and fish especially humans. It should be noted however that cAMP values will generally be lower in cell-free body samples than in body cells, e.g. about nanomolar levels in serum as opposed to micromolar levels in intracellular fluids.

In many biological samples, once removed from the host species the cAMP content reduces rapidly over time. For this reason, the samples used in the assay method of the invention will generally be pretreated to reduce or eliminate cAMP degradation. Typically this may involve addition of a buffer and/or a chelating agent (e.g. EDTA) . For assays of body tissue or organ samples, since cAMP levels can drop very rapidly on sample extraction or on the subject's death or exposure to stress, it may be desirable to freeze the sample as quickly as possible after extraction, e.g. by plunging into liquid nitrogen. The sample may then be processed further, e.g. ground, solvent extracted, etc, before assay performance.

The method of the invention is a method of assaying for cAMP. This may involve generation of a quantitative, semiquantitative or qualitative result, e.g. the concentration of cAMP in the sample, the concentration of cAMP relative to another analyte, allocation of the result to a band in a multi-band representation of cAMP concentration, indication of the cAMP concentration as being above (or below) a predetermined threshold value (e.g. one indicative of a normality/abnormality boundary or a mild/severe boundary), or even an indication of a predicted outcome, e.g. the period for which a whole blood sample may continue to be stored under refrigeration while still being suitable for use in a transfusion. (For the latter, the sample tested will generally be lysed citrated whole blood or red blood cell concentrate). All such determinations of cAMP content are considered to fall within the assay method of the invention.

Besides assaying for cAMP, the assay method of the invention may be used to assay for other cyclic nucleotides (e.g. cGMP) and cyclic nucleotide analogs that are capable of binding to the cAPK cAMP binding site. If such assays are competitive assays, then the competing labelled analyte may be a labelled cAMP or, more preferably, a labelled version of the desired analyte. Such assays are of particular importance in the investigation of the biodistribution and pharmacokinetics of candidate or actual cyclic nucleotide (analog) drug compounds or precursors. Unlike the conventional cAMP assays referred to above, the assay method of the invention is sensitive and accurate enough to detect such compounds. In such assays, if desired, the sample may be treated to reduce or eliminate any cAMP content which might interfere, e.g. by treating the sample with an agent to which cAMP binds but to which the candidate or drug does not or which serves to transform cAMP into a species which does not bind at the cAPK cAMP binding site, e.g. an enzyme with phosphodiesterase activity which serves to transform cAMP (but not the candidate or drug) into a species which does not bind significantly at the cAPK cAMP binding site, or an antibody which binds cAMP but not the candidate or drug.

Examples of cyclic nucleotides and analogs which can be assayed for in this way include 8-aminohexylamino-cAMP, 8-bromo-cAMP, 8-chloro-cAMP, 8-chlorophenylthio-cAMP, N6-monobutyryl-cAMP and cGMP.

Thus viewed from a further aspect the invention provides a method for assaying for a cyclic nucleotide or cyclic nucleotide analog, said method comprising contacting said sample with a polypeptidic binding agent capable of binding said cyclic nucleotide or cyclic nucleotide analog and optionally also with a labelled competitor species capable of binding to said binding agent, and detecting conjugates of said binding agent with said cyclic nucleotide or cyclic nucleotide analog or said competitor species, characterized in that said binding agent comprises functional cAPK cAMP B-binding sites only.

Viewed from a still further assay the invention also provides a kit for an assay for a cyclic nucleotide or cyclic nucleotide analog, said kit comprising a polypeptidic primary binding agent capable of binding said cyclic nucleotide or cyclic nucleotide analog; optionally a labelled competitor species capable of binding to said binding agent; and optionally a secondary binding agent; characterized in that said primary binding agent comprises functional cAPK cAMP B-binding sites only.

This kit and assay method will preferably utilize the features mentioned above in connection with the cAMP assay and kit.

The assays of the invention, unlike the prior art antibody-based assays, are insensitive to the presence of divalent metal ions up to relatively high concentrations (e.g. 10 mM). This is of importance since the labile concentrations of such metal ions (e.g. Ca²⁺ and Mg²⁺) are strongly affected by the presence of the chelating agents which are required to stabilize cAMP concentrations in samples before the assays are performed.

All publications referred to herein are hereby incorporated by reference.

The assay method of the invention will now be described further by reference to the following non-limiting Examples.

### EXAMPLE 1

### cAMP Binding Agent Preparation

The NcoI blunt end fragment of the cDNA for a fusion polypeptide of GST and non-mutagenized human RIα containing a thrombin cleavage site between GST and RIα was inserted in the NcoI-HindIII sites of plasmid pGEX-KG, the insert corresponding to nucleotides 103 to 1474 of the published sequence.

For mutagenesis a QuickChange Site-Directed Mutagenesis Kit (available from Stratagene) was used. The RIα encoding double stranded plasmid and two synthetic oligonucleotide primers with the desired mutations were annealed and extended by means of the Pfu DNA polymerase. The synthetic oligonucleotide primers were 5′-GGAGGGAGCTTTGAAGAACTTGCTTTG and 3′-CCTCCCTCGAAACTTCTTGAACGAAAC. After temperature cycling, the parental DNA template was digested using Dpn 1. The mutated DNA was transformed into Epicurian Coli® XL1-Blue supercompetent cells. The mutations were confirmed by sequencing (ABI Prism 3700).

The plasmid was transformed into E. coli BL21, and preincubated for 2 hours before expression was induced with 0.4 mM IPTG. The bacterial incubation was performed at 25° C.

The expressed GST hRIα fusion protein was purified by binding to glutathione-agarose (Pharmacia), according to the manufacturer's protocol. The fusion protein was further purified on a Superdex 200 gel filtration column (Pharmacia FPLC system). The GST-tag was cleaved off by thrombin followed by an additional FPLC size exclusion chromatography step.

SDS-PAGE mobility of GST-hRIα fusion protein corresponded to the expected value of 81 kDa using bovine serum albumin, hen ovalbumin and Bio-Rad high molecular weight standards for calibration.

The thrombin cleaved protein had SDS-PAGE mobility of 51 kDa.

### EXAMPLE 2

### Assay Performance

Sample Preparation

Male Wistar rats (120-400 g) were anaesthetized, the liver exposed, and a biopsy snap-frozen between the metal clamps of a Wollenberger tong precooled in liquid nitrogen. The frozen tissue was pulverized. (The powder could be stored in liquid nitrogen for months without cAMP degradation). To extract cAMP the powder was precipitated in ice-cold 5% (w/v) aqueous trichloroacetic acid in 0.1 M HCl (1 ml per 50 mg tissue powder), and centrifuged (20,000×g av.) for 10 minutes. The trichloroacetic acid was removed from the supernatant by repeated (4×) extraction with at least 4 volumes of water-saturated diethyl ether. The sample was neutralized by the addition of 20 μl of 10 M NaOH.

Assay Performance

100 μL of sample is mixed with 50 μL of 3 nM ³H-cAMP (Amersham plc, UK) and then with 50 μL of the thrombin cleaved binding agent of Example 1 (1.2 nM in cAMP binding sites) in 50 mM HEPES (adjusted to pH 7.4 with dipotassium phosphate and containing 20 mM EDTA, 3 mM EGTA, 0.5 mg/mL serum albumin, 0.2 mg/mL soybean trypsin inhibitor and 0.5 mM DTE). The mixture is incubated for 2 to 18 hours and then mixed vigorously with 1 mL ice cold 80% saturated ammonium sulphate.

2×2 mL of ice cold 65% saturated ammonium sulphate is passed through a 25 mm diameter membrane filter (0.45 μm pore size, e.g. HAMK available from Millipore) whereafter the precipitated sample is passed through the same filter under suction. Immediately after the sample has run through, 2 mL of ice cold 65% saturated ammonium sulphate are applied followed by a further two rinses with 2 mL of ice cold 65% saturated ammonium sulphate.

The filter is transferred to a scintillation vial containing 1 mL of 2% w/v aqueous sodium dodecyl sulphate solution. The precipitate is dissolved by vortexing for 10 minutes. Thereafter 7 mL of scintillation fluid (“Emulsifier Safe for aqueous samples” from Packard) are added and the vial is counted in a beta-counter for at least 4 minutes.

A calibration curve, prepared using the same assay procedure and standard solutions of cAMP in 50 mM HEPES (adjusted to pH 7.4 with dipotassium phosphate and containing 20 mM EDTA, 3 mM EGTA, 0.5 mg/mL serum albumin, 0.2 mg/mL soybean trypsin inhibitor and 0.5 mM DTE) is used to determine the cAMP content of the sample.

### EXAMPLE 3

### Assay Performance

Where the sample contains a low quantity of cAMP or a high concentration (e.g. 10 mM) of a weakly binding substance (e.g. ATP or AMP) which can dissociate from the binding agent post-incubation, the assay of Example 2 may be performed omitting the ³H-cAMP from the initial incubation and subsequently performing a one hour post incubation in the presence of 10 nM ³H-cAMP. The procedure is otherwise unchanged.

1. A method for assaying for cAMP in a sample, said method comprising contacting a sample with an unknown cAMP content with a polypeptidic cAMP binding agent and optionally with a labelled cAMP and detecting conjugates of cAMP or labelled cAMP and said binding agent, characterized in that said binding agent comprises functional cAPK cAMP B-binding sites only.

2. A method as claimed in claim 1, wherein said binding agent has disabled A-binding sites.

3. A method as claimed in claim 1, wherein said binding site is an RIα B-site.

4. A method as claimed in claim 1, wherein said labelled cAMP is labelled at the 8-position by iodine-125.

5. A method as claimed in claim 1, wherein said labelled cAMP is attached to a substrate surface at the 8-position.

6. A method as claimed in claim 1, wherein said cAMP conjugates are detected using surface plasmon resonance.

7. A method as claimed in claim 1, wherein said labelled cAMP is labelled with tritium.

8. A method as claimed in claim 1, wherein said binding site is capable of binding cAMP with a K_{D} of less than 300% of that of the site in native human cAPK.

9. A method as claimed in claim 1, wherein said binding site is capable of binding cAMP with a K_{D} of less than 110% of that of the site in native human cAPK.

10. A kit for a cAMP assay, said kit comprising a polypeptidic primary binding agent capable of binding cAMP; optionally, a labelled cAMP; and optionally a secondary binding agent; characterized in that said primary binding agent comprises functional cAPK cAMP B-binding sites only.

11. A polypeptidic cAMP binding agent which comprises functional cAPK cAMP B-binding sites only, and compositions and items comprising said binding agent.

12. cAMP labelled at the 8-position by iodine-125, and compositions thereof.

13-14. (canceled)

15. A method for assaying for a cyclic nucleotide or cyclic nucleotide analog, said method comprising contacting said sample with a polypeptidic binding agent capable of binding said cyclic nucleotide or cyclic nucleotide analog and optionally also with a labelled competitor species capable of binding to said binding agent, and detecting conjugates of said binding agent with said cyclic nucleotide or cyclic nucleotide analog or said competitor species, characterized in that said binding agent comprises functional cAPK cAMP B-binding sites only.

16. A kit for an assay for a cyclic nucleotide or cyclic nucleotide analog, said kit comprising a polypeptidic primary binding agent capable of binding said cyclic nucleotide or cyclic nucleotide analog; optionally, a labelled competitor species capable of binding to said binding agent; and optionally a secondary binding agent; characterized in that said primary binding agent comprises functional cAPK cAMP B-binding sites only.
